(19) United States
(12) Patent Application Publication
(10) Pub. No.: US 2024/0197401 A1
(43) Pub. Date: Jun. 20, 2024

(54) METHOD FOR DESIGNING PATIENT-SPECIFIC IMPLANT AND GUIDANCE, PROGRAM AND APPARATUS THEREFOR

(71) Applicant: SeeAnn Solution Co., Ltd., Incheon (KR)

(72) Inventors: Anna SEO, Incheon (KR); Youngjin JEONG, Incheon (KR); Hakjong NOH, Incheon (KR); Chunghwan BAEK, Seoul (KR)

(73) Assignee: SeeAnn Solution Co., Ltd., Incheon (KR)

(21) Appl. No.: 18/316,433

(22) Filed: May 12, 2023

(30) Foreign Application Priority Data
Dec. 19, 2022 (KR) 10-2022-0178706

(51) Int. Cl. A61B 34/10; G06T 19/20

(52) U.S. Cl. CPC A61B 34/10; G06T 19/20; A61B 2034/105; G06T 2219/2004

(57) ABSTRACT

Disclosed are a method for designing a patient-specific implant and guidance, and a program and apparatus therefor. According to the design method of the present disclosure, an area to be cut is defined in a first skeleton image of a patient's skeleton where an affected area is positioned, and an area to be implanted is defined in a second skeleton image of a skeleton of the other area including a graft material.

### CROSS-REFERENCE TO RELATED APPLICATIONS

The present application is based upon and claims the benefit of priority to Korean Patent Application No. 10-2022-0178706, filed on Dec. 19, 2022. The disclosure of the above Korean application is hereby incorporated by reference herein in its entirety.

### BACKGROUND

### 1. Technical Field

The technology disclosed herein relates to a design of a graft material to be implanted into an amputation area of a patient, and a design of guidance for cutting the amputation area or the graft material.

### 2. Related Art

Thanks to the development of medical technology, attempts to replace damaged human body tissues with other body tissues are continuously being made. Typically, when oral cancer occurs in a patient's mandible (lower jaw bone), cutting the bone in the corresponding area is the most obvious treatment. In this case, the bone in the corresponding area is cut, and a part of a skeleton of a suitable shape is cut and implanted from the bone called “fibula” on the side of the shinbone.

Since these surgeries are high risk and even minor errors may cause irreversible damage to the patient, it is helpful to design an area to be cut and an area to be implanted in advance in a virtual 3-dimensional space, and furthermore, it is required to design a guidance that may help mitigate errors during actual bone cutting.

### SUMMARY

The present disclosure is to simulate a transplantation process by specifying a skeleton of an area to be cut and a skeleton of an area to be implanted in a virtual three-dimensional space.

Another object of the present disclosure is to design guidance for fixing an area to be cut in a virtual three-dimensional space so as to reduce errors during actual skeleton cutting.

The technical problems to be achieved by the embodiments of the present disclosure are not limited to the technical problems as described above, and other technical problems may be inferred from the following embodiments.

According to an aspect of the present disclosure, a method performed by an apparatus for designing a patient-specific implant and guidance includes: specifying a first skeleton image of a patient's skeleton on which an affected area where a graft material is to be implanted is positioned; modeling a second skeleton image of a skeleton of another area including the graft material; specifying first spatial information on a cutting target area in the first skeleton image; specifying second spatial information on a region to be implanted in the second skeleton image; and overlapping the second skeleton image and the first skeleton image by matching the first spatial information with the second spatial information.

The specifying of the first skeleton image may include: modeling a skeleton combination image including a plurality of skeleton images adjacent to each other; in a plane penetrating through the skeleton combination image which is movable along a specific axis, specifying a position of the plane on the specific axis; and specifying, as the first skeleton image, a skeleton image penetrating through the plane whose position on the specific axis is specified, among the plurality of skeleton images included in the skeleton combination image.

The specifying of the first spatial information may include: generating one or more planes penetrating through the first skeleton image; selecting a specific closed area corresponding to the area to be cut from among one or more closed areas defined in the first skeleton image by the one or more planes; and determining, as the first spatial information, a set of coordinates specified on at least two cut surfaces of the specific closed area.

The specifying of the second spatial information may include determining, as the second spatial information, a set of coordinates specified by a number corresponding to the number of coordinates included in the first spatial information on the second skeleton image.

The overlapping may include moving the second skeleton image by a difference between each coordinate included in the first spatial information and each coordinate included in the second spatial information.

The design method may further include: applying a change in an angle of at least one of a roll, a yaw, and a pitch to the second skeleton image overlapping the first skeleton image.

The design method may further include: specifying, as the area to be implanted, an area overlapping the area to be cut among the second skeleton images overlapping the first skeleton image.

The area to be cut and the area to be implanted may be displayed with different opacities in a state where the first skeleton image and the second skeleton image are overlapped.

The design method may further include: based on the first skeleton image, designing a first guidance for cutting a skeleton corresponding to the area to be cut from the patient's skeleton.

The designing of the first guidance may include designing a plurality of fixing parts for fixing an actual skeleton to be cut corresponding to the area to be cut in contact with the patient's skeleton. Specifically, the designing of the plurality of fixing parts may include: designing a first sub-fixing unit that is in contact with a cut surface of the area to be cut and orthogonal to a plane defining the area to be cut; and designing a second sub-fixing unit that is in contact with the cut surface of the area to be cut and orthogonal to the first sub-fixing unit. More specifically, the designing of the first sub-fixing unit may include: generating a first cover having a thickness equal to a first offset on a surface of the first skeleton image excluding the area to be cut; and designing a partial area of the first cover that is in contact with the cut surface of the area to be cut as a first sub-fixing part. Meanwhile, the designing of the second sub-fixing unit may include: generating a second cover having a thickness equal to a second offset on a plane defining the area to be cut; separating the second cover into two areas based on a plane defining the area to be cut; selecting an area overlapping the first sub-fixing part from among the two separated areas; and selecting, as a second sub-fixing part, a partial area in contact with the cut surface of the area to be cut in the selected area.

The designing of the first guidance may further include generating a perforated part for screw insertion into at least some of the plurality of fixing parts.

The designing of the first guidance may further include designing a handle part connecting the plurality of fixing parts. Specifically, the designing of the handle part may include: generating a third cover having a thickness equal to a third offset on the surface of the first skeleton image; and designing the handle part by connecting a specified point on at least two of the plurality of fixing parts and one or more specified points in the third cover.

The design method may further include: based on the second skeleton image, designing a second guidance for cutting the graft material from the other skeleton. Specifically, the designing of the second guidance may include designing the second guidance by excluding an area of the partially overlapping second skeleton image from a hexahedron partially overlapping the second skeleton image. More specifically, the designing of the second guidance may include: generating a virtual first hexahedron surrounding the second skeleton image; reducing a volume of the first hexahedron and transforming the first hexahedron into a second hexahedron partially overlapping the second skeleton image; generating a third hexahedron by cutting the second hexahedron into a plane defining the area to be cut; and designing the second guidance by excluding an area of a second skeleton image partially overlapping the third hexahedron in the third hexahedron.

According to another aspect of the present disclosure, an apparatus for designing a patient-specific implant and guidance includes: an input/output interface; a memory that stores an instruction; and a processor, in which the processor is connected to the input/output interface and the memory to specify a first skeleton image of a patient's skeleton on which an affected area where a graft material is to be implanted is positioned, model a second skeleton image of a skeleton of another area including the graft material, specify first spatial information on an area to be cut in the first skeleton image, specify first spatial information on an area to be implanted in the second skeleton image, and overlap the second skeleton image with the first skeleton image by matching the first spatial information with the second spatial information.

Detailed contents of other exemplary embodiments are described in a detailed description and are illustrated in the accompanying drawings.

### BRIEF DESCRIPTION OF THE DRAWINGS

FIG. 1 is a flowchart illustrating a design method according to an embodiment.

FIG. 2 is a diagram illustrating a process of specifying a first skeleton image.

FIG. 3 is a diagram illustrating an area to be cut in the first skeleton image.

FIGS. 4A to 4C are diagrams illustrating the first skeleton image from which the area to be cut is excluded and a modeled second skeleton image.

FIGS. 5A to 5C are diagrams illustrating the first skeleton image and the second skeleton image overlapping each other.

FIGS. 6A to 6G are diagrams illustrating a process of designing a first guidance.

FIGS. 7A to 7C are diagrams illustrating a process of designing a second guidance.

FIG. 8 is a block diagram illustrating a structure of a design apparatus according to an embodiment.

### DETAILED DESCRIPTION

General terms that are currently widely used are selected as terms used in embodiments in consideration of functions in the present disclosure, but may be changed depending on the intention of those skilled in the art or a judicial precedent, the emergence of a new technique, and the like. In addition, in a specific case, terms arbitrarily chosen by an applicant may exist. In this case, the meaning of such terms will be mentioned in detail in a corresponding description portion of the present disclosure. Therefore, the terms used in exemplary embodiments of the present disclosure should be defined on the basis of the meaning of the terms and the contents throughout the present disclosure rather than simple names of the terms.

Throughout the specification, unless otherwise specified, “including” any component means that other components may be further included rather than excluding other components. In addition, terms “˜unit”, “˜module”, and the like, described in the specification refer to a processing unit of at least one function or operation and may be implemented by hardware or software or a combination of hardware and software. Unlike the illustrated example, specific operations may not be clearly distinguished.

The expression “at least one of a, b, and c” described throughout the specification may include “a alone,” “b alone,” “c alone,” “a and b,” “a and c,” “b and c,” or “all of a, b, and c”.

In the following description, terms “transmission”, “communication”, “sending”, “receiving” of signals, messages, or information and other similar terms refer to not only directly transmitting information, messages, or information from one component to another, but also transmitting information, messages, or information from one component to another via other components.

In particular, “transmitting” or “sending” a signal, message or information to a component indicates the final destination of the signals, messages, or information and does not mean a direct destination. The same is true for “reception” of the signals, messages or information. Also, in the present disclosure, two or more data or information being “related” means that when one data (or information) is obtained, at least a portion of other data (or information) can be obtained based thereon.

In addition, the terms such as ‘first’, ‘second’, or the like, may be used to describe various components, but these components are not to be construed as being limited to these terms. The terms are used in order to distinguish one component from another component.

For example, the ‘first’ component may be named the ‘second’ component, and vice versa, without departing from the scope of the present disclosure.

Hereinafter, embodiments of the disclosure will be described in detail with reference to the accompanying drawings so that those skilled in the art to which the disclosure pertains may easily practice the disclosure. However, the disclosure may be implemented in various different forms, and is not limited to embodiments described herein.

Hereinafter, embodiments of the present disclosure will be described in detail with reference to the drawings.

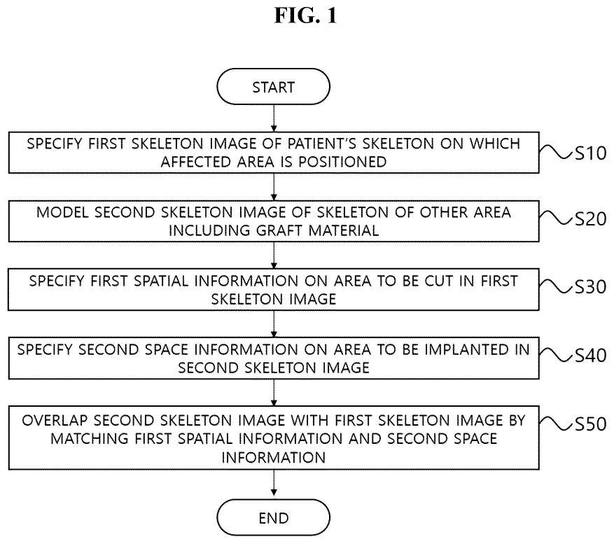

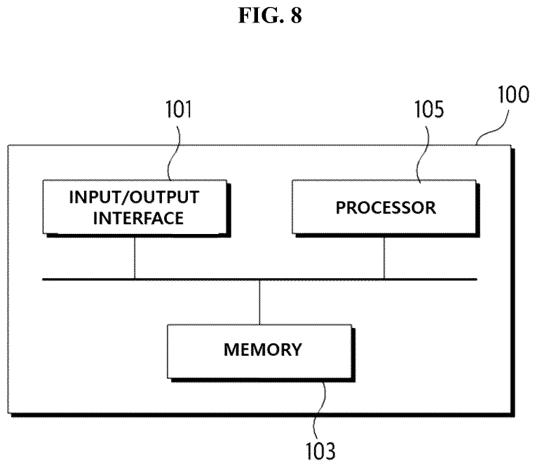

FIG. 1 is a flowchart illustrating a design method according to an embodiment. The method illustrated in FIG. 1 may be exemplarily performed by an electronic apparatus 100 described with reference to FIG. 8. In FIG. 1, a method of the graft material and guidance is described by dividing into a plurality of steps, but at least some of the steps are performed in reverse order, combined with other steps, performed together, omitted, divided into detailed steps. Alternatively, one or more steps not illustrated may be added and performed.

An apparatus (hereinafter referred to as “design apparatus”) for designing a patient-specific implant and guidance specifies a first skeleton image of a patient's skeleton where a graft material is to be implanted (S10). In this case, as an example, “patient's skeleton” may include a mandible (lower jawbone) of a facial part where an affected area is positioned.

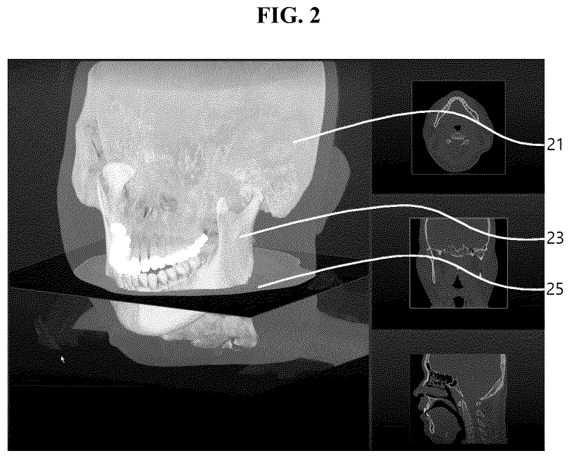

In this regard, referring to FIG. 2 illustrating a process of specifying the first skeleton image, in the embodiment, the design apparatus may model skeleton combination images 21 and 23 including a plurality of skeleton images adjacent to each other, specify a position of a plane 25 on a specific axis in the plane 25 penetrating through skeleton combination images 21 and 23 that are movable along a specific axis, and specify, among the plurality of skeleton images included in the skeleton combination images 21 and 23, a skeleton image 23 penetrating through the plane 25 whose position on a specific axis is specified as the first skeleton image.

The design apparatus models a second skeleton image of a skeleton of the other area including the graft material (S20). In this case, exemplarily, the “skeleton of the other area” may include a fibula of a patient or another person.

The design apparatus specifies first spatial information on an area to be cut in the first skeleton image (S30).

The design apparatus specifies second spatial information on an area to be implanted in the second skeleton image (S30).

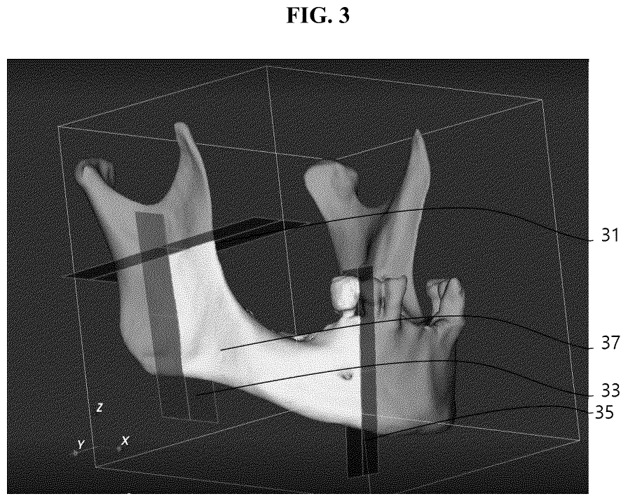

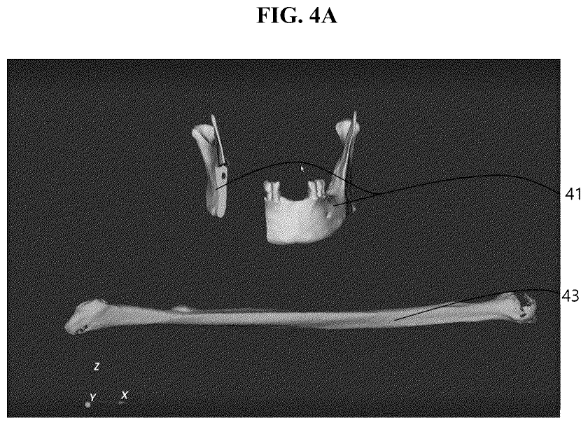

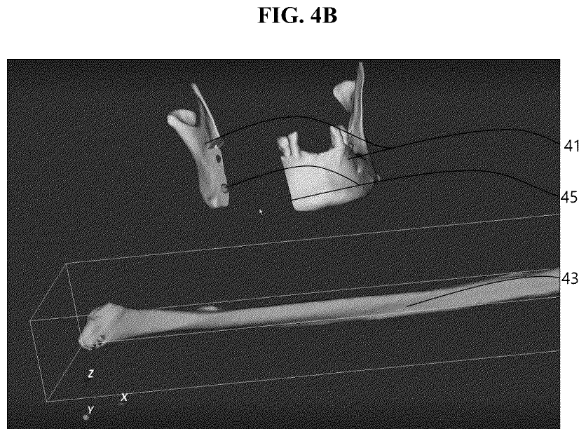

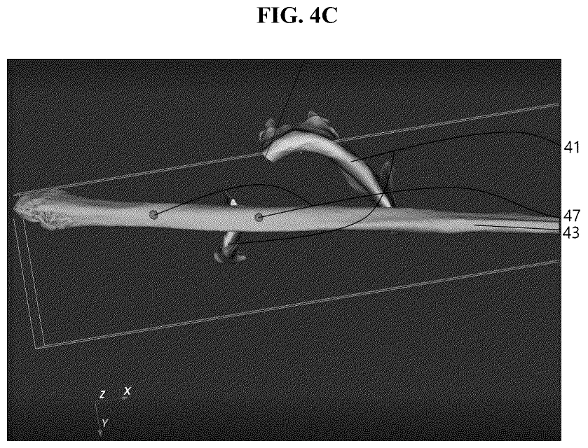

In this regard, referring to FIGS. 3, 4a, 4b, and 4c, in the embodiment, the design apparatus may generate one or more planes 31, 33, and 35 penetrating through the first skeleton image 23, and select a specific closed area 37 corresponding to the area to be cut from among one or more closed areas defined in the first skeleton image 23 by the one or more planes 31, 33, and 35 to determine, as the first spatial information, a set 45 of coordinates each specified on at least two of cut planes of the specific closed area 37 in the first skeleton image 41 from which the specific closed area 37 is excluded.

In addition, in the embodiment, the design apparatus may determine, as the second spatial information, a set 47 of coordinates specified as a number corresponding to the number of coordinates included in the first spatial information 45 on the second skeleton image 43.

The design apparatus overlaps the second skeleton image with the first skeleton image by matching the first spatial information and the second spatial information (S50).

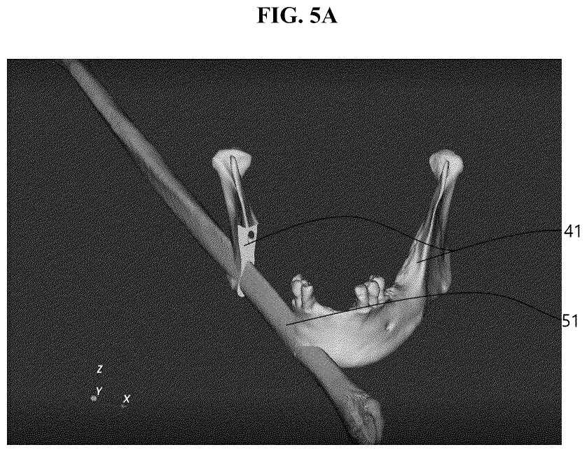

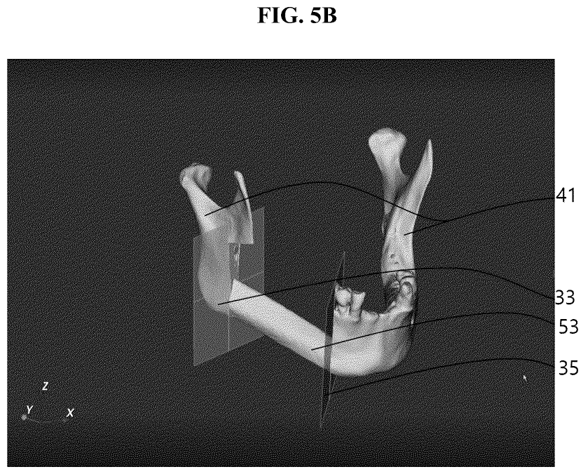

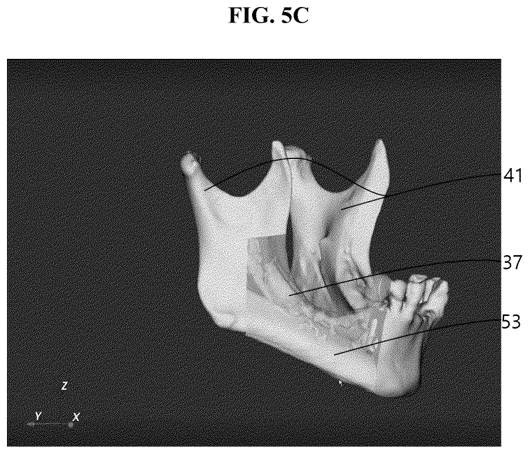

In this regard, referring to FIGS. 5A to 5B illustrating the first skeleton image and the second skeleton image overlapping each other, in an embodiment, the design apparatus may move the second skeleton image 43 by a difference between each coordinate included in the first spatial information 45 and each coordinate included in the second spatial information 47 to overlap the first skeleton image 41 and the second skeleton image 51 (since the movement of the second skeleton image is a coordinate transformation, the reference number is changed from 43 to 51). In addition, the design apparatus may apply a change in an angle of at least one of roll, yaw, and pitch to the overlapping second skeleton image 51 for detailed position adjustment of the overlapping second skeleton image 51. Meanwhile, the design apparatus may specify an area 53 in which the overlapping second skeleton image 51 is cut into one or more planes 33 and 35 as an area 53 to be implanted, and according to the embodiment, specify an area overlapping the area to be cut among the overlapping second skeleton images 51 as the area to be implanted. In both cases, the specified areas to be implanted are the same, and it is significant that the area to be implanted may be specified through various methods.

Meanwhile, referring to FIG. 5C, after the area 53 to be implanted is specified, the design apparatus may overlap and display the specific closed area (area to be cut) 37 and the area 53 to be implanted along with the first skeleton image 41, but display the two areas with different opacities.

However, although not illustrated in the drawings, depending on the shape of the first skeleton image to be replaced by surgery or the like, two or more fragments of a skeleton of the other area to be implanted into the corresponding area may be inserted into a graft material. In this case, the second skeleton image may include two or more fragment images of the second skeleton.

Meanwhile, as an additional embodiment, the design apparatus may additionally design a first guidance for cutting the skeleton corresponding to the area to be cut in the patient's skeleton based on the first skeleton image.

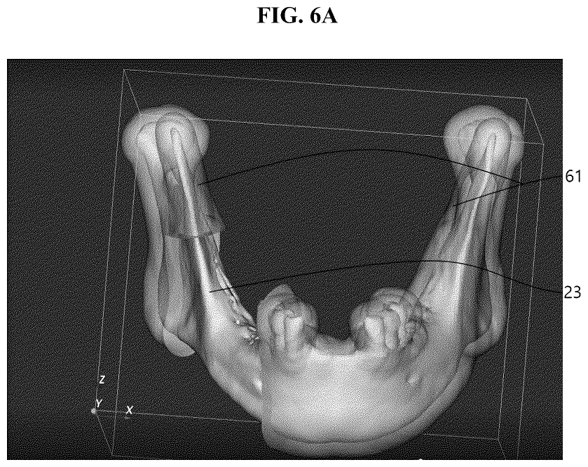

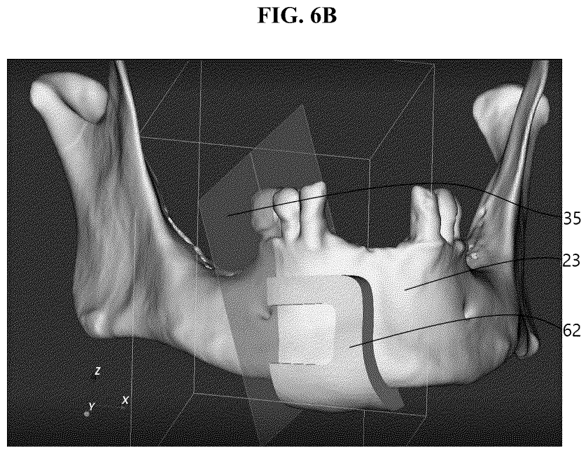

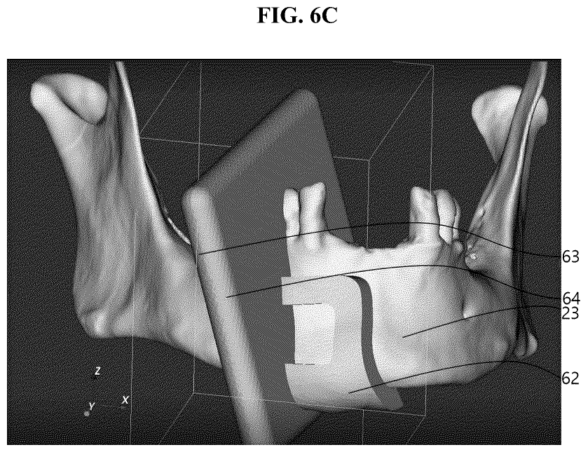

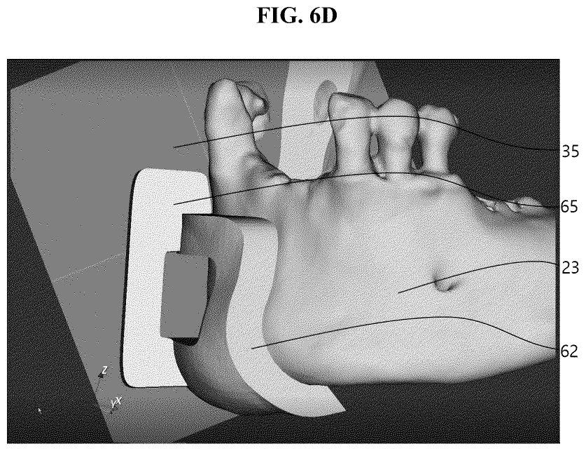

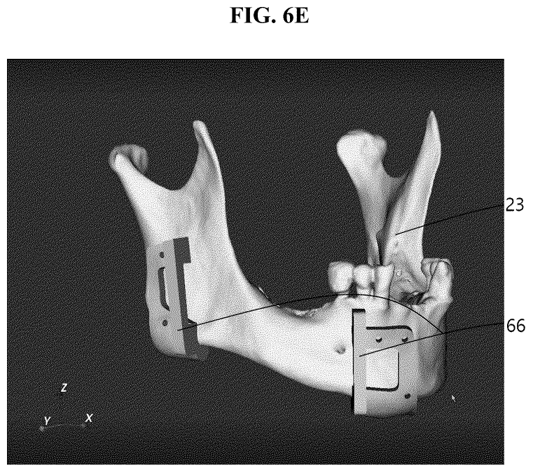

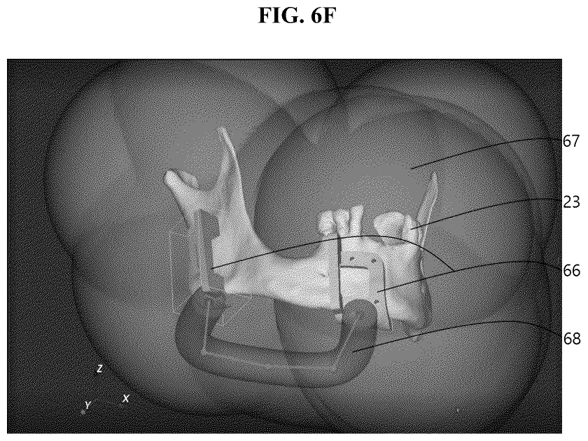

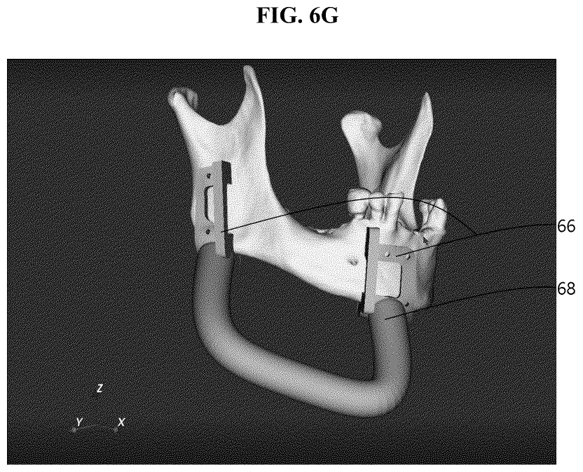

In an embodiment, as a process of designing the first guidance, the design apparatus may design a plurality of fixing parts for fixing the actual skeleton to be cut corresponding to the area 37 to be cut in contact with the patient's skeleton. Specifically, the design apparatus may design a first sub-fixing part and a second sub-fixing part as some elements constituting each fixing part. The first sub-fixing part may be in contact with the cut surface of the area 37 to be cut and may be orthogonal to the plane defining the area to be cut, and the second sub-fixing part may be in contact with the cut surface of the area 37 to be cut and may be orthogonal with the first sub-fixing part. A process of designing the first sub-fixing part and the second sub-fixing part will be described below in more detail with reference to FIGS. 6A to 6D.

To design the first sub-fixing part, the design apparatus may generate a first cover 61 having a thickness equal to a first offset on a surface of the first skeleton image excluding the area 37 to be cut, and may design a portion of the first cover 61 contacting the cut surface of the area 37 to be cut as the first sub-fixing part 62. In addition, to design the second sub-fixing part, the design apparatus may generate a second cover having a thickness equal to a second offset in the plane 35 defining the area 37 to be cut, separate the second cover into two areas 63 and 64 based on the plane 35 defining the area 37 to be cut, and select the area 64 overlapping the first sub-fixing part among the two separated areas 63 and 64 to select a partial area in contact with the cut surface of the area 37 to be cut as the second sub-fixing part 65 in the selected area 64.

Meanwhile, the design apparatus may generate a perforated part for screw insertion into at least some of the plurality of designed fixing parts. FIG. 6E shows that a perforated part is formed on the surface of the first sub-fixing part 62, but the perforated part may be formed not only on the first sub-fixing part 62 but also on the second sub-fixing part 65 according to the embodiment.

Meanwhile, in an embodiment, as an additional process of designing the first guidance, the design apparatus may design a handle part connecting the plurality of fixing parts. Specifically, referring to FIG. 6F, the design apparatus may generate a third cover 67 having a thickness equal to a third offset on the surface of the first skeleton image 23, and design a handle part 68 by connecting a specified point on at least two of the plurality of fixing parts 66 and one or more specified points in the third cover 67. FIG. 6G illustrates an exemplary shape of the first guidance comprising the fixing part 66 and the handle part 68 thus designed. A user may position the actually cut skeleton between the fixing parts of the first guidance manufactured according to the design, and separate the cut skeleton from the entire patient's skeleton by cutting an end of the cut skeleton based on the fixing part. In this case, since the height of the handle part is considerable, the user may effectively fix the fixing part without touching a nerve existing in a surgical area of a patient.

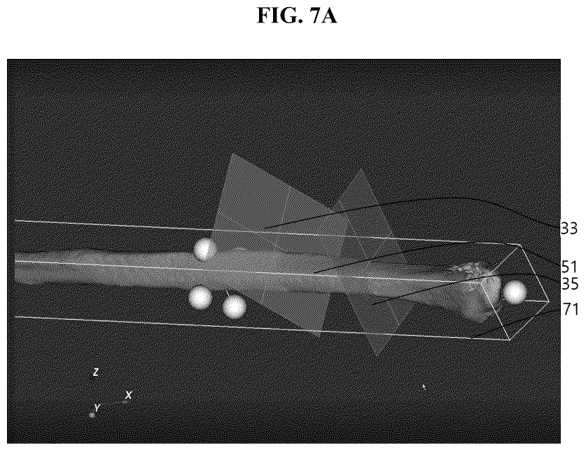

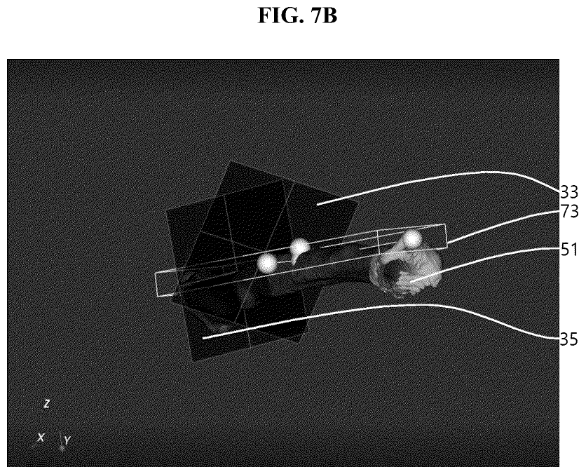

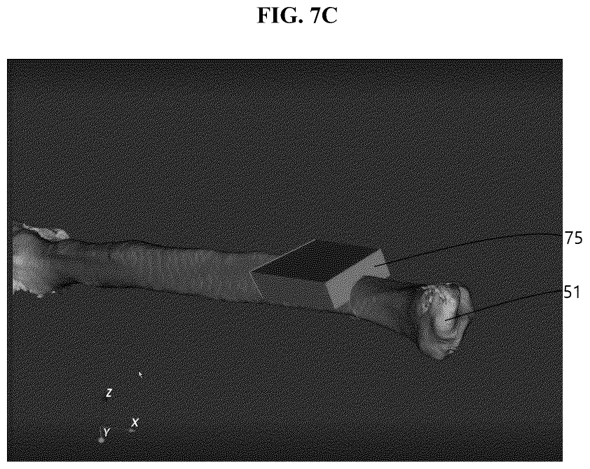

On the other hand, as an additional embodiment, the design apparatus may additionally design a second guidance for cutting the graft material at the skeleton of the other area based on the second skeleton image. Specifically, the design apparatus may design the second guidance by excluding the area of the partially overlapping second skeleton image from a hexahedron partially overlapping the second skeleton image. For example, as illustrated in FIGS. 7A and 7C, the design apparatus may generate a virtual first hexahedron 71 surrounding the second skeleton image 51, reduce the volume of the first hexahedron 71 and transform the first hexahedron 71 into a second hexahedron 73 partially overlapping the second skeleton image 51, generate the third hexahedron by cutting the second hexahedron 73 into the planes 33 and 35 defining the area 37 to be cut, and design the second guidance 75 by excluding the area of the second skeleton image partially overlapping the third hexahedron in the third hexahedron. When the second skeleton image includes two or more second skeletal fragment images, one or more grooves for cutting the second skeleton into fragments may be formed in the second guidance 75. Also, like the first guidance, the perforated part for screw insertion may be formed on the second guidance 75.

FIG. 8 is a block diagram illustrating a structure of a design apparatus according to an embodiment.

The electronic apparatus 100 may include an input/output interface 101, a memory 103, and a processor 105 according to an embodiment. In one embodiment, the electronic apparatus 100 may server or database through be connected to an external a transceiver or a communication interface, and exchange data.

The processor 105 may perform at least one method described above through FIGS. 1 to 7C. The memory 103 may store information for performing at least one method described above through FIGS. 1 to 7C. The memory 103 may be a volatile memory or a nonvolatile memory.

The processor 105 may control the electronic apparatus 100 for executing a program and providing information. A code of the program executed by the processor 105 may be stored in the memory 103.

The processor 105 may be connected to the memory 103 to specify the first skeleton image of the patient's skeleton where the affected area where the graft material is to be implanted is positioned, model the second skeleton image for the skeleton of the other area including the graft material, specify the first spatial information on the area to be cut in the first skeleton image, specify the second spatial information on the area to be implanted in the second skeleton image, and overlap the second skeleton image and the first skeleton image by matching the first spatial information and the second spatial information.

In the electronic apparatus 100 illustrated in FIG. 8, only components related to the present embodiment are illustrated. Accordingly, it can be understood by those of ordinary skill in the art related to the present embodiment that other general-purpose components may be further included in addition to the components illustrated in FIG. 8.

The apparatus according to the above-described embodiments may include a processor, a memory that stores and executes program data, a permanent storage such as a disk drive, a communication port that communicates with an external device, a touch panel, a key, a user interface device such as a button, and the like. Methods implemented as software modules or algorithms may be stored on a computer-readable recording medium as computer-readable codes or program instructions executable on the processor. Here, examples of the computer-readable recording medium may include magnetic storage media (for example, a read-only memory (ROM), a random-access memory (RAM), a floppy disk, a hard disk, etc.), optical reading media (for example, a CD-ROM or a digital versatile disc (DVD)), and the like. The computer-readable recording medium may be distributed in computer systems connected to each other through a network, and as a result, the computer-readable codes may be stored in a distributed scheme and executed. The medium may be readable by a computer, stored in a memory, and executed on a processor.

The present embodiments may be represented by functional block configurations and various processing steps. These functional blocks may be implemented by various numbers of hardware and/or software components that execute specific functions. For example, the embodiment may employ integrated circuit configurations, such as memory, processing, logic, and a look-up table, capable of executing various functions by control of one or more microprocessors or other control devices. Similar to executing the components in software programming or software elements, and the present embodiment can be implemented in programming or scripting languages such as python, C, C++, Java, and assembler, including various algorithms implemented by a combination of data structures, processes, routines or other programming configurations. Functional aspects may be implemented in algorithms executed on one or more processors. In addition, the present embodiment may employ a conventional technology for electronic environment setting, signal processing, message processing, and/or data processing, and the like. Terms such as “mechanism,” “element,” “means,” and “configuration” may be used widely, and are not limited to mechanical and physical configurations. The terms may include the meaning of a series of routines of software in connection with a processor or the like.

According to the method disclosed herein, it is possible to risks in actual bone graft surgery by knowing a correct cutting method, cutting position, and the like of a skeleton in advance.

In addition, by designing a guidance to intuitively grasp the area to be cut by contacting a patient's skeleton according to the method disclosed herein, it is possible to control errors that may occur depending on a difference in environment in a virtual 3D space and a real space.

Effects of the present invention are not limited to the above-mentioned effects, and other effects that are not mentioned will be clearly understood by those skilled in the art from the description of the claims.

The above-described embodiments are merely examples, and other embodiments may be implemented within the scope of the following claims.

What is claimed is:

1. A method performed by an apparatus for designing a patient-specific implant and guidance, the method comprising:
specifying a first skeleton image of a patient's skeleton on which an affected area where a graft material is to be implanted is positioned;
modeling a second skeleton image of a skeleton of an other area including the graft material;
specifying first spatial information on an area to be cut in the first skeleton image;
specifying second spatial information on a region to be implanted in the second skeleton image; and
overlapping the second skeleton image and the first skeleton image by matching the first spatial information with the second spatial information.

2. The method of claim 1, wherein the specifying of the first skeleton image includes:
modeling a skeleton combination image including a plurality of skeleton images adjacent to each other;
in a plane penetrating through the skeleton combination image which is movable along a specific axis, specifying a position of the plane on the specific axis; and
specifying, as the first skeleton image, a skeleton image penetrating through the plane whose position on the specific axis is specified, among the plurality of skeleton images included in the skeleton combination image.

3. The method of claim 1, wherein the specifying of the first spatial information includes:
generating one or more planes penetrating through the first skeleton image;
selecting a specific closed area corresponding to the area to be cut from among one or more closed areas defined in the first skeleton image by the one or more planes; and
determining, as the first spatial information, a set of coordinates specified on at least two cut surfaces of the specific closed area.

4. The method of claim 1, wherein the specifying of the second spatial information includes determining, as the second spatial information, a set of coordinates specified by a number corresponding to the number of coordinates included in the first spatial information on the second skeleton image.

5. The method of claim 1, wherein the overlapping includes moving the second skeleton image by a difference between each coordinate included in the first spatial information and each coordinate included in the second spatial information.

6. The method of claim 1, further comprising:
applying a change in an angle of at least one of a roll, a yaw, and a pitch to the second skeleton image overlapping the first skeleton image.

7. The method of claim 1, further comprising:
specifying, as the area to be implanted, an area overlapping the area to be cut among the second skeleton images overlapping the first skeleton image.

8. The method of claim 1, wherein the area to be cut and the area to be implanted are displayed with different opacities in a state where the first skeleton image and the second skeleton image are overlapped.

9. The method of claim 1, further comprising:
based on the first skeleton image, designing a first guidance for cutting a skeleton corresponding to the area to be cut from the patient's skeleton.

10. The method of claim 9, wherein the designing of the first guidance includes designing a plurality of fixing parts for fixing an actual skeleton to be cut corresponding to the area to be cut in contact with the patient's skeleton.

11. The method of claim 10, wherein the designing of the plurality of fixing parts includes:
designing a first sub-fixing unit that is in contact with a cut surface of the area to be cut and orthogonal to a plane defining the area to be cut; and
designing a second sub-fixing unit that is in contact with the cut surface of the area to be cut and orthogonal to the first sub-fixing unit.

12. The method of claim 11, wherein the designing of the first sub-fixing unit includes:
generating a first cover having a thickness equal to a first offset on a surface of the first skeleton image excluding the area to be cut; and
designing a partial area of the first cover that is in contact with the cut surface of the area to be cut as a first sub-fixing part.

13. The method of claim 11, wherein the designing of the second sub-fixing unit includes:
generating a second cover having a thickness equal to a second offset on a plane defining the area to be cut;
separating the second cover into two areas based on a plane defining the area to be cut;
selecting an area overlapping the first sub-fixing part from among the two separated areas; and
selecting, as a second sub-fixing part, a partial area in contact with the cut surface of the area to be cut in the selected area.

14. The method of claim 10, wherein the designing of the first guidance further includes designing a handle part connecting the plurality of fixing parts.

15. The method of claim 14, wherein the designing of the handle part includes:
generating a third cover having a thickness equal to a third offset on the surface of the first skeleton image; and
designing the handle part by connecting a specified point on at least two of the plurality of fixing parts and one or more specified points in the third cover.

16. The method of claim 1, further comprising:
based on the second skeleton image, designing a second guidance for cutting the graft material from the other skeleton.

17. The method of claim 16, wherein the designing of the second guidance includes designing the second guidance by excluding an area of the partially overlapping second skeleton image from a hexahedron partially overlapping the second skeleton image.

18. The method of claim 17, wherein the designing of the second guidance includes:
generating a virtual first hexahedron surrounding the second skeleton image;
reducing a volume of the first hexahedron and transforming the first hexahedron into a second hexahedron partially overlapping the second skeleton image;
generating a third hexahedron by cutting the second hexahedron into a plane defining the area to be cut; and
designing the second guidance by excluding an area of a second skeleton image partially overlapping the third hexahedron in the third hexahedron.

19. A computer-readable recording medium on which a program for performing the method according to claim 1 is recorded.

20. An apparatus for designing a patient-specific implant and guidance, the apparatus comprising:
an input/output interface;
a memory that stores an instruction; and
a processor,
wherein e processor is connected to the input/output interface and the memory to
specify a first skeleton image of a patient's skeleton on which an affected area where a graft material is to be implanted is positioned,
model a second skeleton image of a skeleton of another area including the graft material,
specify first spatial information on an area to be cut in the first skeleton image,
specify first spatial information on an area to be implanted in the second skeleton image, and
overlap the second skeleton image with the first skeleton image by matching the first spatial information with the second spatial information.
